(19) United States
(12) Patent Application Publication
(10) Pub. No.: US 2017/0281918 A1
(43) Pub. Date: Oct. 5, 2017

(54) Nasal Dilator

(71) Applicant: Erich Gross, Santa Maria, CA (US)

(72) Inventor: Erich Gross, Santa Maria, CA (US)

(21) Appl. No.: 15/476,048

(22) Filed: Mar. 31, 2017

Related U.S. Application Data
(60) Provisional application No. 62/315,772, filed on Mar. 31, 2016.

(51) Int. Cl. A61M 29/00; A61F 2/82

(52) U.S. Cl. CPC A61M 29/00; A61F 2/82; A61M 2210/0618; A61F 2230/0069

(57) ABSTRACT

A nasal dilator. The nasal dilator includes a hollow, tubular body having open first and second ends that allows air to pass through. The tubular body is adapted to be positioned within a user's nostril, or nasal passageway, to maintain or increase the capacity to breathe through one's nostrils. The tubular body includes apertures positioned at intervals that allow hair and other nasal objects to extend within the tubular body. These apertures provide comfort in use. The nasal dilator remains within the nostril by friction fit, and may be selectively utilized in either, or both nostrils.

### CROSS REFERENCE TO RELATED APPLICATION

This application claims the benefit of U.S. Provisional Application No. 62/315, 772 filed on Mar. 31, 2016. The above identified patent application is herein incorporated by reference in its entirety to provide continuity of disclosure.

### BACKGROUND OF THE INVENTION

### FIELD OF THE INVENTION

The present invention relates to nasal dilators. More specifically, the present invention provides a nasal dilator having a tubular body with an open first end in fluid communication with an open second end. The tubular body is hollow and dimensioned to be received by a nostril and includes a circular cross-section that extends along an entire length thereof. One or more traverse apertures disposed on the tubular body allow hair and other nasal objects to not interfere with the comfort of utilizing the nasal dilator.

Many individuals have nostrils that are relatively small and decreases the airflow through the nasal passageway. As a result, those individuals may experience shortness of breath, or discomfort when attempting to breathe directly through one's nostrils. Other individuals may experience head colds or other medical complications that decrease the capacity to breathe through one's nostrils uninhibited. The diminishing breathing capacity adversely affects performance in aerobic activities, such as sports.

Devices have been disclosed in the known art that relate to nasal dilators. These include devices that have been patented and published in patent application publications. These devices in the known art have several known drawbacks. For example, one such device provides a nasal insert that is inserted either simultaneously into both nostrils via a connector therebetween, or with a clip. These devices however fail to provide for a nasal dilator that remains in the nostril via friction fit and without the need for uncomfortable fasteners.

In light of the devices disclosed in the known art, it is submitted that the present invention substantially diverges in design elements from the devices in the known art and consequently it is clear that there is a need in the art for an improvement to existing nasal dilator devices. In this regard the instant invention substantially fulfills these needs.

### SUMMARY OF THE INVENTION

In view of the foregoing disadvantages inherent in the known types of nasal dilators now present in the art, the present invention provides a new and improved nasal dilator wherein the same can be utilized for providing convenience for the user when the user is running or exercising and benefits from increase breathing capacity.

It is therefore an object of the present invention to provide a new and improved nasal dilator having a tubular body with an open first and an open second end that facilitates the flow of air therethrough. The tubular body maintains or forces the nasal passageway open to increase the user's ability to breathe effectively.

It is another object of the present invention to provide a nasal dilator with one or more apertures disposed on the tubular body at intervals, wherein the apertures provide comfort to the user by permitting hair and other objects therethrough.

Another object of the present invention is to provide a nasal dilator having a cylindrical, non-tapered tubular body that remains in a user's nostril via friction fit.

Another object of the present invention is to provide a nasal dilator that may be readily fabricated from materials that permit relative economy and are commensurate with durability.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of the nasal dilator.

FIG. 2 shows a cross-sectional view of the nasal dilator taken along line 2-2 of FIG. 1.

### DETAILED DESCRIPTION OF THE INVENTION

Reference is made herein to the attached drawings. Like reference numerals are used throughout the drawings to depict like or similar elements of the nasal dilator. For the purposes of presenting a brief and clear description of the present invention, the preferred embodiment will be discussed as used for insertion in the nostrils. The figures are intended for representative purposes only and should not be considered to be limiting in any respect.

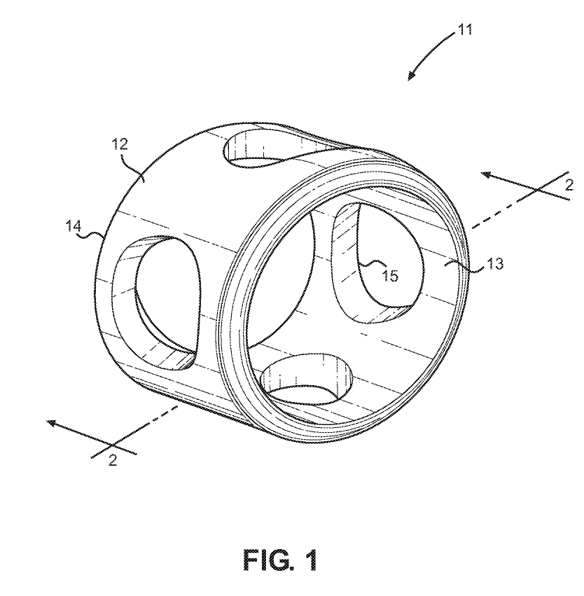

Referring to FIG. 1, there is shown a perspective view of the nasal dilator. The nasal dilator 11 maintains a user's nostrils in an expanded position when worn during exercise or other activities. The nasal dilator 11 comprises a tubular body 12 having an open first end 13 and an open second end 14, wherein the tubular body 12 is hollow and the first and second ends 13, 14 are in fluid communication with each other. In the shown embodiment, the tubular body 12 is dimensioned to be received by a nostril, such that deformation of the nostril during the course of breathing is not lessened, thereby permitting a greater volume of air to be inhaled by the user. In one embodiment and use, the nasal dilator 11 is adapted to be inserted into a user's nostrils, and be secured therein via a friction fit.

The tubular body 12 includes one or more traverse apertures 15 disposed thereon. In the shown embodiment, there are four traverse apertures 15 disposed wholly on the tubular body 12. However, in alternative embodiments, the tubular body 12 may include many apertures 15 disposed in many configuration, such as staggered, or terminating partially in the open first end 13 and the open second end 14. The one or more traverse apertures 15 are each disposed equidistant from the open first end 13 and the open second end 14. The apertures 15 are configured to allow hair and other nasal objects to extend into the tubular body 12. In this way, the nasal dilator 11 allows the natural movement of nasal objects while worn and does not cause discomfort to a user when worn. Further, the edges of the open first end 13, the open second end 14, and the one or more apertures 15 are smooth. However, in alternative embodiments, the edges may be beveled, or comprise any other shape and dimension.

In the shown embodiment, the nasal dilator 11 may be composed of a plastic. However, in alternative embodiments, the nasal dilator 11 may be constructed from any material suitable for nasal insertion. In one embodiment, the tubular body 12 is rigid, such that once inserted into the nostril, the nostril cannot constrict the flow of air therethrough. In another embodiment, the nasal dilator 11 is semi-flexible, providing pliable properties when under force, and that are biased to return to the un-deformed shape upon removal of force.

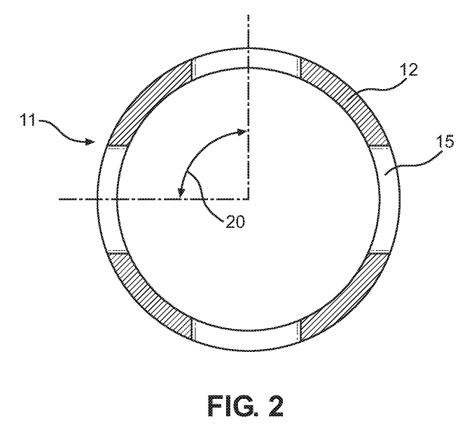

Referring to FIG. 2, there is shown a cross-sectional view of the nasal dilator taken along line 2-2 of FIG. 1. In the shown embodiment, the tubular body 12 has a circular cross-section that extends along an entire length thereof. The traverse apertures 15 are positioned equidistant from adjacent apertures. In the shown embodiment, the tubular body 12 includes four traverse apertures 15 positioned ninety degrees from each other when viewed along line 2-2. In alternative embodiments, the angle 20 between adjacent traverse apertures 15 may range between zero and one hundred and eighty degrees.

In one embodiment, the tubular body 12 comprises a thickness of no more than two millimeters when viewed along line 2-2. However, in alternative embodiments, the thickness may be equal to or greater than two millimeters. Further, in the shown embodiment, each aperture 15 has a diameter of five millimeters. Alternatively, the diameter of each aperture may be different from the other apertures, and the diameters may have any dimension. In this way, the nasal dilator 11 is suitable for individuals, wherein nostril dimension may vary.

In one use, a first nasal dilator 11 is used in conjunction with a substantially similar nasal dilator 11. The nasal dilator 11 is configured for usage in either nostril, and may be used independently of a second substantially similar nasal dilator 11.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A nasal dilator, comprising:
a tubular body including an open first end in fluid communication with an open second end;
wherein the tubular body is hollow and dimensioned to be received by a nostril;
one or more traverse apertures disposed annularly about the tubular body;
wherein the tubular body has a uniform circular cross-section that extends along an entire length thereof.

2. The nasal dilator of claim 1, wherein the tubular body is rigid.

3. The nasal dilator of claim 1, wherein the tubular body comprises a thickness of no more than two millimeters.

4. The nasal dilator of claim 1, wherein the one or more traverse apertures comprise four apertures, each disposed equidistant from the open first end and the open second end.

5. The nasal dilator of claim 4, wherein each aperture is disposed ninety degrees from an adjacent aperture.

6. The nasal dilator of claim 5, wherein each aperture has a diameter of five millimeters.

7. The nasal dilator of claim 1, wherein the edges of the open first end, the open second end, and the one or more apertures are smooth.
